(19) United States
(12) Patent Application Publication
(10) Pub. No.: US 2008/0153849 A1
(43) Pub. Date: Jun. 26, 2008

(54) Process for the Preparation of Crystalline (6Rrs)-N(5)-Formyl-5,6,7,8-Tetrahydrofolic Acid

(76) Inventors: Francese Giancarlo, Montagnola (CH); Moreno Morosoli, Tesserete (CH)

Correspondence Address: BINGHAM MCCUTCHEN LLP, Three Embarcadero Center, San Francisco, CA, 94111-4067, US

(21) Appl. No.: 10/589,751

(22) PCT Filed: Feb. 18, 2005
(86) PCT No.: PCT/CH05/00092

§ 371 Date: Aug. 17, 2006

(30) Foreign Application Priority Data
Feb. 20, 2004 (CH) 285/04

(51) Int. Cl. A61K 31/4985; C07D 475/04; A61P 7/06

(52) U.S. Cl. 514/255.05; 544/258

(57) ABSTRACT

The inventive method for the production of crystalline (6RS)-N(5)-formyl-5,6,7,8-tetrahydrofolic acid or amorphic (6S)-N(5)-formyl-5,6,7,8-tetrahydrofolic acid is characterized in that an aqueous solution of (6RS)- or (6S)-calcium folinate, which has a temperature of 40° C. to 50° C., and an aqueous solution of hydrochloric acid or acetic acid are added to stirred water having a temperature of 2° C. to 12° C., such that the temperature is kept at 2° C. to 12° C. in the mixture thus obtained when the two above-mentioned solutions are added and the pH value is kept at 2,5 to 3,5, the solid thus arising is isolated by means of filtration or centrifugation, the solid is initially washed with cold water and then with an aqueous organic solvent, and the washed solid, i.e. crystalline (6RS)-N(5)-formyl-5,6,7,8-tetrahydrofolic acid or amorphous (6S)-N(5)-formyl-5,6,7,8-tetrahydrofolic acid is dried at reduced pressure and obtained.

The present invention is directed to a process for the preparation of crystalline (6RS)-N(5)-formyl-5,6,7,8-tetrahydrofolic acid or of amorphous (6S)-N(5)-formyl-5,6,7,8-tetrahydrofolic acid.

The present invention is also directed to crystalline (6RS)-N(5)-formyl-5,6,7,8-tetrahydrofolic acid and to amorphous (6S)-N(5)-formyl-5,6,7,8-tetra-hydrofolic acid as such.

The present invention is also directed to a process for the preparation of a concentrated, stable solution, especially of an injection solution or of an infusion solution, of the sodium or potassium salt of (6RS)- or (6S)-folinic acid as well as to this solution as such.

N(5)-Formyl-5,6,7,8-tetrahydrofolic acid is also named folinic acid.

The pharmacological meaning of the well soluble alkali metal salts of reduced folates is described in the beginning of the specification of EP 0 667 159.

The known prior art for the preparation of folinic acid is described in NO 172 492.

With this reference are deemed to be disclosed also herein the statements as made in these two references.

It is to be supposed that the folinic acid prepared according to these processes is in amorphous form.

Crystalline folinic acid has not been disclosed until to the filing date of the present invention.

In example 1 of WO 93/17022 is described the preparation of pure (6RS)-folinic acid. In this example is mentioned a “precipitate” of (6RS)-folinic acid.

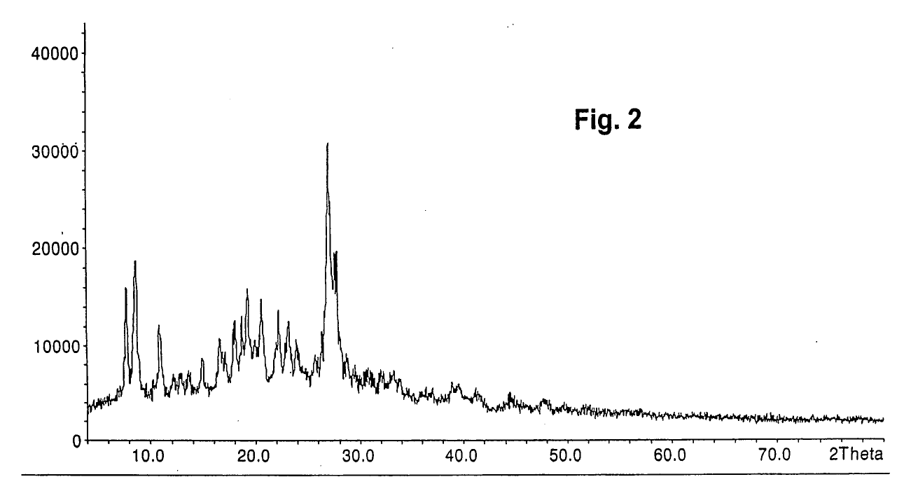

From the X-ray analysis of the product obtained according to this example and as shown in FIG. 2 it is obvious that this product contains at least 30% of amorphous (6RS)-folinic acid.

One would expect that folinic acid would be obtainable by direct acidification of an aqueous solution of a water soluble folinate salt.

When for example an aqueous solution of calcium folinate is acidified with diluted hydrochloric acid then is obtained an untreatable, rubber like product, and even when various parameters, such as temperature, concentration, reaction time, are varied.

According to example 6 of EP 0 293 029 there is added carefully diluted hydrochloric acid to an aqueous solution of calcium-(6S)-folinate, whereby the (6S)-folinic acid should precipitate and should be obtainable by filtration.

The applicant of the present invention could not repeat this working example: it was obtained always an untreatable, rubber like product, despite the fact that various parameters, such as temperature, concentration, reaction time, were varied.

According to E. Khalifa, A. N. Ganguly, J. H. Bieri and M. Viscontini, Helv. Chim. Acta, Vol. 63, 2554 (1980) the herein described folinic acid is clearly a mixture of the (6RS)-diastereoisomers and is in amorphous form.

Therefore, in the working example, that is described in EP 0 667 159, amorphous (6RS)-folinic acid has been used.

It is well known that the biological active form of the reduced folates has the (6S)-configuration; see for example E. E. van Tamelen, R. E. Hopla, Journal of the American Chemical Society, 101, 6114-6115, (1979).

It follows therefrom that the injection solution as described in EP 0 667 159 contains 50% of inactive substance with the (6R)-configuration. This in turn means that the human body to which is administered such an injection solution is unnecessarily burdened (double the length of administration and administration of an inactive substance).

It is an object of the present invention to overcome the above mentioned drawbacks.

It is a further object of the present invention to provide crystalline (6RS)-folinic acid and amorphous (6S)-folinic acid.

There shall also be provided a process for the preparation of these two compounds.

There shall also be provided a concentrated, stable solution of the sodium or potassium salt of (6S)-folinic acid.

There shall also be provided a process for the preparation of this solution, starting from amorphous (6S)-folinic acid.

With the present invention these objects are met.

It has been found now quite surprisingly that crystalline (6RS)-folinic acid is obtained when one proceeds according to the teachings of the characterizing part of claim 1.

It also has been found that the amorphous (6S)-folinic acid prepared according to the teachings of the characterizing part of claim 1 has a stability comparable with the crystalline (6RS)-folinic acid.

The inventive process for the preparation of crystalline (6RS)-N(5)-formyl-5,6,7,8-tetrahydrofolic acid or of amorphous (6S)-N(5)-formyl-5,6,7,8-tetra-hydrofolic acid

is characterized in that there is added to stirred water having a temperature from 2° C. to 12° C. simultaneously
an aqueous solution having a temperature from 40° C. to 50° C. of (6RS)- or of (6S)-calcium-folinate, and
an aqueous solution of hydrochloric acid or of acetic acid

in such a way that in the obtained mixture during the addition of both of said solutions on one hand the temperature is kept at a value from 2° C. to 12° C. and on the other hand the pH value is kept at a value from 2.5 to 3.5,

the formed solid is isolated by means of filtration or centrifugation,

this solid is washed first with cold water and then with an aqueous organic solvent, and

the washed solid, that is crystalline (6RS)-N(5)-formyl-5,6,7,8-tetrahydrofolic acid or amorphous (6S)-N(5)-formyl-5,6,7,8-tetrahydrofolic acid, is dried under reduced pressure and is obtained.

The inventive process for the preparation of a concentrated, stable solution, especially of an injection solution or of an infusion solution, of the sodium or potassium salt of (6RS)- or (6S)-folinic acid,

is characterized in that crystalline (6RS)-folinic acid or amorphous (6S)-folinic acid is suspended in water, that is degassed and that is acceptable for the preparation of injection solutions or of infusion solutions, at room temperature under an inert gas atmosphere, then

an aqueous solution of sodium or potassium hydroxide, -hydrogencarbonate or -carbonate is added in portions during such a long time until a clear solution is formed having the respective desired pH value,

the obtained solution is subjected to a sterile filtration, and

the obtained sterile solution is filled into vials or into ampoules under an inert gas atmosphere.

The inventive concentrated, stable solution, especially an injection solution or an infusion solution, is characterized in that it contains beside water either (6S)-sodium-folinate or (6S)-potassium-folinate.

This inventive solution may be used
for the preparation of a medicament for rescues—rescue agent—after the treatment with high doses of methotrexate, or
for the preparation of a medicament which is combined with 5-fluorouracil, or
for the preparation of a medicament for the treatment of megaloblastic anemia and dihydropteridin reductase deficiency.

Preferred embodiments of the present invention are defined in the dependent claims.

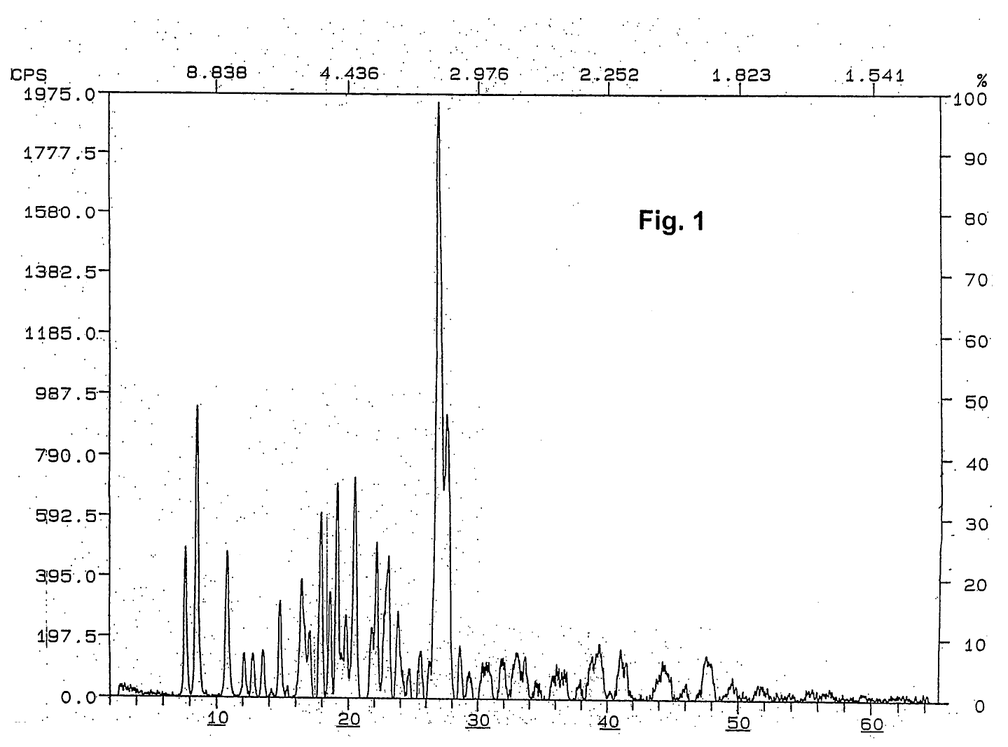

FIG. 1 shows an X-ray analysis of inventive crystalline (6RS)-folinic acid.

It is obvious from FIG. 1 that the compound (6RS)-folinic acid is highly crystalline.

It has been noted quite surprisingly that the (6S)-N(5)-formyl-5,6,7,8-tetrahydrofolic acid prepared according to the teachings of the characterizing part of claim 1 is not available in crystalline form. The corresponding X-ray analysis shows no significant peaks.

The following examples illustrate the present invention.

### EXAMPLE 1

250 g of (6RS)-Calcium-folinate, prepared according to NO 172 492, were dissolved in 3.3 liters of deionized water at a temperature of 55° C.

This clear solution (in the following part denoted as solution A) was cooled to a temperature of 50° C.

In a 10 liter reaction vessel with cooling and stirring devices were added 2.52 liters of deionized water and were cooled under stirring to a temperature of 6° C.

To this 6° C. cold water were added simultaneously under stirring said solution A and 18% aqueous hydrochloric acid by means of two peristaltic pumps.

The velocity of the addition of the solution A was chosen such that the temperature in the obtained mixture remained between 6° C. and 10° C.

The relative velocity of the addition of the 18% aqueous hydrochloric acid was chosen such that the pH value in the obtained mixture remained between 2.8 und 3.2.

The addition of the solution A and of the hydrochloric acid was finished after 3 hours.

The obtained suspension was stirred for 1 additional hour at a temperature from 6° C. to 10° C.

The formed crystalline solid was isolated by means of centrifugation.

The solid was washed once with deionized water and once with a 9:1 mixture (v/v) of acetone and deionized water. Both washing solutions had a temperature from 5° C. to 10° C.

The washed, crystalline solid was dried under reduced pressure (20 to 30 mbar) at room temperature during 2 hours and at a temperature of 50° C. during 1 hour.

There were obtained 215 g of crystalline (6RS)-folinic acid.

HPLC-purity: 99.6%

HPLC-assay/content: 100.3%

It follows from FIG. 1 that the obtained solid was crystalline.

This solid was stored at a temperature from 2° C. to 8° C. in a refrigerator. Thereby the following stability dates were obtained for this solid:

| Time (months) | Assay (HPLC) | Purity (HPLC) |
|---|---|---|
| 0 | 99.4% | 99.78% |
| 3 | 99.0% | 99.79% |
| 6 | 99.1% | 99.75% |
| 9 | 100.2% | 99.82% |
| 12 | 99.9% | 99.79%. |

### EXAMPLE 2

290 g of (6S)-Calcium-folinate, prepared according to EP 600 460 and NO 172 492, were dissolved in 4.9 liters of deionized water at a temperature of 58° C.

This clear solution (in the following part denoted as solution B) was cooled to a temperature of 45° C.

In a 20 liter reaction vessel with cooling and stirring devices were added 4 liters of deionized water and were cooled under stirring to a temperature of 6° C.

To this 6° C. cold water were added simultaneously under stirring said solution B and 18% aqueous hydrochloric acid by means of two peristaltic pumps.

The velocity of the addition of the solution B was chosen such that the temperature in the obtained mixture remained between 6° C. and 10° C.

The relative velocity of the addition of the 18% aqueous hydrochloric acid was chosen such that the pH value in the obtained mixture remained between 2.8 und 3.2.

The addition of the solution B and of the hydrochloric acid was finished after 3 hours.

The obtained suspension was stirred for 1 additional hour at a temperature from 6° C. to 10° C.

The formed amorphous solid was isolated by means of centrifugation.

The solid was washed once with deionized water and once with a 94:6 mixture (v/v) of ethanol and deionized water. Both washing solutions had a temperature from 5° C. to 10° C.

The washed, amorphous solid was dried under reduced pressure (20 to 30 mbar) at room temperature during 2 hours.

There were obtained 146 g of amorphous (6S)-folinic acid.

HPLC-purity: 99.2%

HPLC-assay/content: 99.0%

Die diastereoisomeric purity was 99.7% (HPLC).

This solid was stored at a temperature from 2° C. to 8° C. in a refrigerator. Thereby the following stability dates were obtained for this solid:

| Time (months) | Assay (HPLC) | Purity (HPLC) |
|---|---|---|
| 0 | 99.0% | 99.2% |
| 1 | 98.5% | 99.0% |
| 2 | 98.1% | 98.9% |
| 3 | 98.9% | 98.9% |
| 6 | 98.1% | 98.4% |
| 12 | 97.8% | 97.2%. |

### EXAMPLE 3

100 g of amorphous (6S)-folinic acid, prepared according to example 2, were suspended in 1.2 liters of degassed, sterile water under a nitrogen atmosphere at room temperature.

Then was added drop wise under stirring a 10% aqueous sodium hydroxide solution during such a long time until a clear solution has been formed which had a pH value of 8.0.

The obtained clear solution was diluted by the addition of degassed, sterile water to a volume of 1.8 liters.

This diluted solution was subjected to a sterile filtration (pore size 0.2 micrometers).

The obtained sterile filtrate was filled under a nitrogen atmosphere into 10 ml sized glass vials.

These glass vials were stored at a temperature from 2° C. to 8° C. in a refrigerator. Thereby the following stability dates were obtained for solution filled into the glass vials:

| Time (months) | Assay (HPLC) | Purity (HPLC) |
|---|---|---|
| 0 | 100.0% | 98.4% |
| 1 | 101.0% | 98.4% |
| 3 | 106.0% | 98.3% |
| 6 | 110.9% | 98.9% |
| 9 | 108.0% | 98.1% |
| 12 | 111.7% | 97.6%. |

After 12 months the solution was still clear; no precipitates could be detected.

1. A process for the preparation of crystalline (6RS)-N(5)-formyl-5,6,7,8-tetrahydrofolic acid or of amorphous (6S)-N(5)-formyl-5,6,7,8-tetrahydrofolic acid, characterized in that there is added to stirred water having a temperature from 2° C. to 12° C. simultaneously
an aqueous solution having a temperature from 40° C. to 50° C. of (6RS)- or of (6S)-calcium-folinate, and
an aqueous solution of hydrochloric acid or of acetic acid
in such a way that in the obtained mixture during the addition of both of said solutions on one hand the temperature is kept at a value from 2° C. to 12° C. and on the other hand the pH value is kept at a value from 2.5 to 3.5,
the formed solid is isolated by means of filtration or centrifugation,
this solid is washed first with cold water and then with an aqueous organic solvent, and
the washed solid, that is crystalline (6RS)-N(5)-formyl-5,6,7,8-tetrahydrofolic acid or amorphous (6S)-N(5)-formyl-5,6,7,8-tetrahydrofolic acid, is dried under reduced pressure and is obtained.

2. The process according to claim 1, characterized in that the stirred water, to which said two solutions are added simultaneously, has a temperature from 6° C. to 10° C.

3. The process according to claim 1, characterized in that the aqueous solution of (6RS)-calcium-folinate has a concentration from 7.5% by weight to 8.5% by weight.

4. The process according to one of claim 1, characterized in that the aqueous solution of (6S)-calcium-folinate has a concentration from 3.0% by weight to 3.7% by weight, preferably 3.5% by weight.

5. The process according to one of claim 1, characterized in that the aqueous solution of (6RS)- or of (6S)-calcium-folinate has a temperature of 46° C.

6. The process according to one of claim 1, characterized in that the aqueous solution of hydrochloric acid has room temperature and has a concentration from 10% by weight to 20% by weight, preferably 18% by weight.

7. The process according to one of claim 1, characterized in that in the obtained mixture during the simultaneous addition of both of said solutions the temperature is kept at a value from 6° C. to 10° C.

8. The process according to one of claim 1, characterized in that in the obtained mixture during the simultaneous addition of both of said solutions the pH value is kept at a value from 2.8 to 3.2.

9. The process according to one of claim 1, characterized in that after the realized simultaneous addition of both of said solutions the obtained mixture is stirred for 1 additional hour at a temperature from 6° C. to 10° C.

10. The process according to claim 1, characterized in that
in the case of the use of (6RS)-calcium-folinate as starting material the formed crystalline solid is washed after the washing with cold water with a 9:1 mixture (v/v) of acetone and water, and that
in the case of the use of (6S)-calcium-folinate as starting material the formed amorphous solid is washed after the washing with cold water with a 94:6 mixture (v/v) of ethanol and water.

11. Crystalline (6RS)-N(5)-formyl-5,6,7,8-tetrahydrofolic acid and amorphous (6S)-N(5)-formyl-5,6,7,8-tetrahydrofolic acid.

12. Crystalline (6RS)-N(5)-formyl-5,6,7,8-tetrahydrofolic acid and amorphous (6S)-N(5)-formyl-5,6,7,8-tetrahydrofolic acid according to claim 11, characterized in that these two compounds have been prepared according to the process according to claim 1.

13. Use of crystalline (6RS)-N(5)-formyl-5,6,7,8-tetrahydrofolic acid or of amorphous (6S)-N(5)-formyl-5,6,7,8-tetrahydrofolic acid for the preparation of an aqueous solution of the sodium or potassium salt of (6RS)- or (6S)-folinic acid.

14. A process for the preparation of a concentrated, stable solution, especially of an injection solution or of an infusion solution, of the sodium or potassium salt of (6RS)- or (6S)-folinic acid,
characterized in that crystalline (6RS)-folinic acid or amorphous (6S)-folinic acid is suspended in water, that is degassed and that is acceptable for the preparation of injection solutions or of infusion solutions, at room temperature under an inert gas atmosphere, then an aqueous solution of sodium or potassium hydroxide, -hydrogencarbonate or -carbonate is added in portions during such a long time until a clear solution is formed having the respective desired pH value,
the obtained solution is subjected to a sterile filtration, and
the obtained sterile solution is filled into vials or into ampoules under an inert gas atmosphere.

15. The process according to claim 14, characterized in that the crystalline (6RS)-folinic acid or the amorphous (6S)-folinic acid is prepared according to the process according to claim 1.

16. The process according to claim 14, characterized in that said clear solution contains from 2% by weight to 15% by weight, especially from 2% by weight to 6% by weight, preferably 5% by weight, of (6RS)- or (6S)-sodium-folinate or of (6RS)- or (6S)-potassium-folinate.

17. The process according to claim 14, characterized in that said clear solution has a pH value in the range from 7.5 to 8.5, especially from 7.9 to 8.1, preferably 8.0.

18. Concentrated, stable solution, especially an injection solution or an infusion solution, characterized in that it contains beside water either (6S)-sodium-folinate or (6S)-potassium-folinate.

19. Solution according to claim 18, characterized in that it is prepared according to the process according to claim 1.

20. Solution according to claim 18, characterized in that it contains from 2% by weight to 15% by weight, especially from 2% by weight to 6% by weight, preferably 5% by weight, of (6S)-sodium-folinate or (6S)-potassium-folinate.

21. Solution according to claim 18, characterized in that it has a pH value in the range from 7.5 to 8.5, especially 7.9 to 8.1, preferably 8.0.

22. Solution according to claim 18, characterized in that it contains neither a stabilizer nor a complexing agent.

23. Solution according to claim 18, characterized in that it is filled into vials or into ampoules having in their interior an inert gas atmosphere, especially a nitrogen atmosphere.

24. Vials or ampoules, characterized in that there is filled into them a concentrated, stable solution according to claim 18.

25. Use of the solution according to claim 18 for the preparation of a medicament for rescues—rescue agent—after the treatment with high doses of methotrexate.

26. Use of the solution according to claim 1 for the preparation of a medicament which is combined with 5-fluorouracil.

27. Use of the solution according to claim 18 for the preparation of a medicament for the treatment of megaloblastic anemia and dihydro-pteridin reductase deficiency.
